(19) United States
(12) Patent Application Publication
(10) Pub. No.: US 2014/0081295 A1
(43) Pub. Date: Mar. 20, 2014

(54) Microadhesive Mesh And Sutures

(71) Applicant: Novo Contour, Inc., Edmonds, WA (US)

(72) Inventors: Michael P.H. Lau, Edmonds, WA (US); Leonard Pease, Bountiful, UT (US)

(21) Appl. No.: 14/027,115

(22) Filed: Sep. 13, 2013

Related U.S. Application Data
(60) Provisional application No. 61/701,439, filed on Sep. 14, 2012.
(60) Provisional application No. 61/801,730, filed on Mar. 15, 2013.

(51) Int. Cl. A61F 2/00

(52) U.S. Cl. CPC A61F 2/0077
USPC 606/151

(57) ABSTRACT

An adhesive surface comprises at least a nanoparticle coating, a metallic coating, or a fiber with a micro/nano patterned topology. The adhesive surface is adhesive to tissue in a living body. The nanoparticle coating may be metallic. In some embodiments, the adhesive surface constitutes one or more elements of a bandage or surgical mesh, is a surface of a suture, or is sized, shaped, and configured for surgical application in a body. The surface or fiber may further comprise or be encased in a dissolvable coating, such as a polysaccharide, oligosaccharide, or sucrose. The fiber may have a surface that is patterned in topological relief, such as ridges in various orientations, cylindrical protrusions substantially normal the fiber surface, undulations of the fiber surface, or random asperities of the fiber surface. The patterning may be less than 700 nm or greater than 10 microns.

### CROSS-REFERENCES TO RELATED APPLICATION

This application claims the benefit of U.S. Provisional Application Nos. 61/701,439, filed Sep. 14, 2012, and 61/801,730, filed Mar. 15, 2013, the disclosures of which are incorporated by reference herein in their entirety.

### TECHNICAL FIELD

This present disclosure relates to anchors used in vivo to secure surgical mesh, tissue supports, tissue scaffolds, stents, tissue replacements, bandages, and sutures. The disclosure specifically provides a novel solution to a long standing need for mesh that durably anchors without hooks or sutures in the field of surgical treatment of organ prolapse, urinary incontinence, and related maladies. The disclosure provides improvements over “tension free” mesh that relies on friction to hold it in place.

### BACKGROUND

Urinary incontinence and pelvic floor disorders adversely affect millions of women, leading to embarrassment, incapacitating falls, and nursing home admission. In the child bearing years, damage to fascia, a connective tissue, and muscles in the pelvic floor during delivery leads to stress urinary incontinence (SUI), a particular form of incontinence characterized by loss of urine upon jumping, coughing, sneezing, or other physical exertion. SUI incidence peaks during midlife and accounts for 55.4% of incontinence in the US. Later in life, incontinence leads to over 50% of nursing facility admissions, and pelvic organ prolapse becomes more significant as urogenital organs lose mass. In net, 30 to 40 percent of women in the US suffer from some form of incontinence, and nearly one in six women will be affected by SUI over their lifetimes. Almost 10 percent of women will receive surgical treatment for urinary incontinence and pelvic organ prolapse at least once in their lifetime. Approximately 180,000 surgeries for SUI and one-half million surgeries for pelvic organ prolapse are performed each year in the US.

State-of-the-art surgical mesh may be secured by one of two primary means. These means are termed anchors or anchorings herein. First, traditionally mesh has been anchored by suturing it in place. The sutures ligate the mesh to adjacent fascia, ligaments, tendons, and other connective tissue and may be absorbable or non-absorbable by the surrounding tissue. The advantage of sutures is that they are durable and prevent the mesh from migrating away from the site of attachment intended by the surgeon. However, suturing requires more extensive surgical dissection than alternative methods to expose the tissue for anchoring, necessitating a larger incision and increasing the risk of bleeding and collateral damage. Also, associated incisions can locally traumatize tissue by disturbing the extracellular matrix and the cells that reside therein, and require addition surgical time compared to alternative techniques. Therefore, open surgery requires more patient recovery time than newer, less invasive surgical strategies. Furthermore, once sutured or anchored, the tension of the mesh can obviously no longer be adjusted. The alternative technology in this product space is mesh that is held in place by friction with adjacent tissue. This mesh is often referred to as “tension free” because it does not have to be tensioned using sutures. This mesh may be inserted directly (i.e., without sutures), significantly simplifying the surgical procedure. However, they can, in some cases, slip either during the surgical procedure or during weeks of patient recovery, causing the failure to achieve the surgical objectives. Here again, it is difficult during surgery to adjust the tension of the mesh using this tension free approach. Both strategies (i.e., sutures and tension free mesh) are currently used to secure mesh in clinical settings.

### SUMMARY

As shown in Table 1, there is a clear need for mesh anchoring technologies that provide both rapid deployment in clinical settings with minimal tissue trauma and superior retention of the mesh placement, both initially and during surgical recovery. This application discloses an improved anchor composition to provide both requirements, enabled by cutting edge micro/nano fabrication and manufacturing techniques. The engineered compositions do not require hooks or sutures, providing an improvement over state-of-the-art methods and compositions. The composition also provides superior adhesion properties and durability compared to polymeric mesh without topographical relief or the disclosed modifications. The anchors are designed to stay in place for multiple weeks so that an extracellular matrix (collagen, fibrinin, elastin, fibroblasts, etc.) can grow through the mesh to provide in vivo native anchoring.

TABLE 1
Mesh Anchoring Properties Including Rate of Mesh Deployment

| | Somewhat | |
|---|---|---|
| | Robust Placement | Very Robust Placement |
| Slow Deployment | Undesirable | Sutures |
| Rapid Deployment | Tension Free Mesh | The Disclosed Invention |

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A-1D illustrate diagrams of fiber with surface topological patterning to anchor the fiber onto tissue, wherein FIG. 1A illustrates fiber displaying topological surface patterning; FIG. 1B illustrates fiber displaying metallic nanoparticles; FIG. 1C illustrates fiber displaying surface topological patterning coated with nanoparticles; and FIG. 1D illustrates fiber decorated with polyglycans or oligosaccharides.

FIGS. 2A and 2 illustrate examples of micro/nano surface patterning, wherein FIG. 2A illustrates high throughput roll-to-roll fiber patterning and decoration strategy to achieve the embodiment shown in FIG. 1C, and FIG. 2B illustrates micro/nano patterning from polymer extrusion at large Deborah numbers. (Adapted from Polymer Processing Fundamentals by Tim A. Osswald, section 3.1, page 55 [1].)

FIG. 3A illustrates a long mesh without gold nanoparticles that clearly sustained much less weight than the mesh with metallic nanoparticles.

FIG. 3B illustrates the mesh with gold nanoparticles embedded in its surface which sustained 80 g of weight and then released completely over 30.9 s.

FIG. 3C illustrates mixing the metallic nanoparticles into the bulk of the PDMS before incubation which decreases the amount of metallic nanoparticle near the surface but dramatically increases the volume of the PDMS mesh with corresponding increases in the surface area.

FIGS. 4A and 4B illustrate an adhesive comprising patterning and/or particles/coatings, which is connected to other surgical implements including, but not limited to sutures, mesh, stents, bandages and the like.

### DETAILED DESCRIPTION

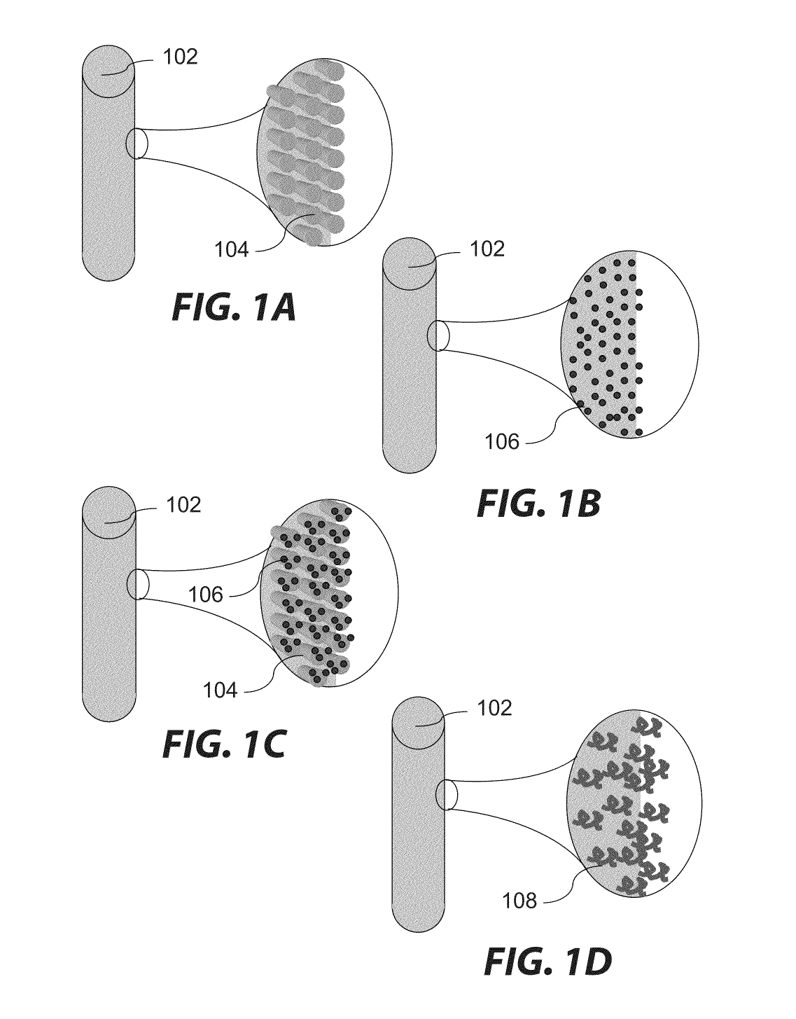

Disclosed herein is an innovative anchoring system for surgical mesh, tissue supports, tissue scaffolds, stents, tissue replacements, bandages, and sutures. In at least one aspect, the mesh comprises patterning at least some of the fibers in relief with nanometer to millimeter length scales (see FIGS. 1A-1D). The fibers are micropatterned and/or nanopatterned using emerging or state-of-the-art fabrication and manufacturing techniques. In other aspects, the anchoring is an element or component of a larger surgical implement designed to adhere to tissue or other implements. In at least one aspect, the surgical implements including, but not limited to, mesh or mesh anchorings comprise coating with metallic particles or layers to enhance binding strength.

For example, surgical implements in the form of fibrous mesh or sutures may be secured to tissue at the macro or micro scale. At the macro scale, clinical practice achieves strategic placement of the mesh using knots, loops or hooks. At the micro to nanoscale, interfacial forces between the fiber and tissue are the primary means of securing the mesh.

For example, tension free mesh uses frictional forces to secure the mesh. However, state-of-the-art commercial mesh consists of smooth fibers that make surgical insertion straightforward and rapid but are non-ideal for securing the mesh. Clinically, this leads to slippage of the mesh relative to the tissue, and, thus, failure to achieve the surgical objectives of tissue support, and may even lead to surgical complications.

Embodiments of the invention described herein include, but are not limited to, surgical implements, mesh, or sutures comprising one or more fibers or bodies with predominantly cylindrical symmetry. The disclosed embodiments illustrate an improved means of securing the fibers to tissue by tuning both the composition and surface morphology of the fibers. The mesh described herein can be deployed though a small incision, through endoscopic means, or by tunneling with blunt dissection. The mesh or suture can also be fabricated in a tailored shape, size, and configuration to meet the clinical needs of surgery. For example, the self-adhering mesh described herein can be cut into a desirable size and shape, and then be inserted through a laparoscopic port. The mesh can thus be deployed without the need for complex tasks of suturing, for example in accomplishing surgery for laparoscopic sacrocolpopexy, in which the vaginal vault is suspended from the surface of the sacrum for vaginal prolapse. In some embodiments, the suturing is minimized or completely eliminated. In other embodiments, the predominate geometry is not cylindrical but comprises longitudinal, Cartesian, or spherical or other geometries. In yet other embodiments, the surgical implements comprise multiple layers with microadhesive properties (i.e., surface patterns and compositions disclosed herein) on any layers that comprise outer layer or lie within proximity to outer layer during the lifetime of the implement.

In at least one embodiment, one or more fibers 102 or one or more surfaces of a surgical implement are patterned in topological relief 104 (see FIGS. 1A-1D). For example, the topological patterning may comprise ridges parallel to the fiber axis, ridges perpendicular to the fiber axis, ridges that spiral around the fiber, cylindrical protrusions 104 normal to the fiber surface, undulations of the fiber surface, random asperities of the fiber surface, setae, et cetera. In various embodiments, the protruding cylinders, undulations and asperities have aspect ratios greater than 100, 30, 10, 3, or 1. In some embodiments, the fibers are composed of biomedical polymers that are of biomedical grade and are clinically acceptable.

In at least one embodiment, the spacing between features is greater than the size of macrophages or their filopodia so that these and other immune cells other can remove bacteria. In various embodiments, the spacing between centers of the patterning is greater than 1, 2, 3, 4, 5, 6, 7, 8, 9, 10, 11, 12, 13, 14, 15, 16, 17, 18, 19, or 20 microns. In at least one embodiment, the spacing between features is smaller than the size of bacteria so that the fibers do not act as bacterial reservoirs that can serve to simulate infection post implantation. In various embodiments, the spacing between centers of patterning is less than 3.0, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, or 0.1 microns.

Intuitively one would expect that it would be best to make a nanohook, but at subcellular sizes in dynamic environments where tissues remodel with regularity, this strategy is less effective [2]. Furthermore, nanohooks are difficult to manufacture. In contrast, the disclosed invention is straightforward to manufacture by adapting a variety of nanomanufacturing techniques known to those skilled in the art.

The purpose of nanopatterning is, at least in part, to enhance the binding and/or adhesion of the mesh to the tissue via surface forces. Patterning the surface increases the total surface area, thereby, increasing the total surface forces between the two objects so that these forces can effectively secure the mesh to the tissue. Fibers comprised of materials that are soft and/or flexible are important to maximize total contact area and, thereby, the adhesion between the two objects. Inflexible surface patterning merely decreases the available contact area with patterning to other inflexible surfaces [3]. The primary forces acting between the mesh and tissue are, inter alia, van der Waals forces, charge-charge interactions, and molecular-specific binding. In completely or predominantly dry environments, van der Waals forces dominate.

This is the primary force responsible for dry adhesion, such as in the adhesion of gecko feet to surfaces [4, 5]. See also the disclosures in U.S. Pre-Grant Publication Nos. 2009/0076241 A1; 2011/0104430 A1; 2011/0117321 A1; 2008/0171836 A1; 2010/0137903 A1; and US 2008/0169059 A1; and U.S. Pat. No. 7,943,703 B2; all of which are incorporated by reference herein.

In liquid or hydrated environments (in contrast to completely or predominantly dry environments, e.g., US 2011/0117321 A1), multiple forces govern surface adhesion. This is in large measure because van der Waals forces are substantially reduced in liquid environments. The composite Hamaker constant is approximately A₁₀₂=(A₁₁^{1/2}−A₀₀^{1/2})(A₂₂^{1/2}−A₀₀^{1/2}), were Aᵢᵢ represent the Hamaker constants for individual materials, 1 and 2 represent two (dissimilar) materials, here the tissue and the mesh, respectively, and 0 represents the intervening media [6, 7].

The Hamaker constant for water ranges between 3.2·10⁻²⁰ J to 3.8·10⁻²⁰ J and that of air or vacuum is considered to be negligible [8-10]. Individual and composite Hamaker constants of approximately the same value or less than k_{b}T=4.1·10⁻²¹ J are considered to be weak as thermal motion is greater than or equal to the attraction. In contrast, gecko adhesion of polystyrene to glass through air is considered to be strong, where A₁₀₂=9.5·10⁻²⁰ J. The reported values of Hamaker constants for tissue and cell masses vary considerably in the literature, from 8.2·10⁻²¹ J to 1.4·10⁻¹⁹ J [11, 12]. Polymers range in the literature from approximately 3.8·10⁻²⁰ J to 9.5·10⁻²⁰ J [13], giving values of A₁₀₂=−1.2·10⁻²⁰ J to 2.5·10⁻²⁰ J. Not only are these values smaller than those for gecko adhesion but in some cases the composite Hamaker constant is negative, which signifies that the van der Waals forces are not attractive but repulsive. Therefore, strategies that are designed for dry environments alone may be weak at best and completely ineffective at worst. This explains the paucity of gecko glue or gecko adhesive strategies reported and/or commercially available for aqueous environments.

In at least one embodiment, the fiber or implement topology is formed into the fibers at the time of initial fabrication. For example, the fiber or implement topology can be made by micro/nano molding processes similar to processes used to make compact disks. Here, the polymer above its glass transition temperature or melting temperature is introduced to a mold that is patterned in relief. The mold is closed forcing the polymer to conform to the relief. The temperature is then quenched below its glass transition temperature. The patterned fiber or surgical implement is released from the mold displaying the desired surface patterning

In at least one embodiment, the polymer is photopolymerizable. Here, the monomer above its glass transition temperature or melting temperature is introduced to a mold that is patterned in relief. The mold comprises an optically transparent material. The mold is closed forcing the monomer to conform to the relief. The monomer is then exposed to photon flux such that it polymerizes and retains the shape of the mold upon release.

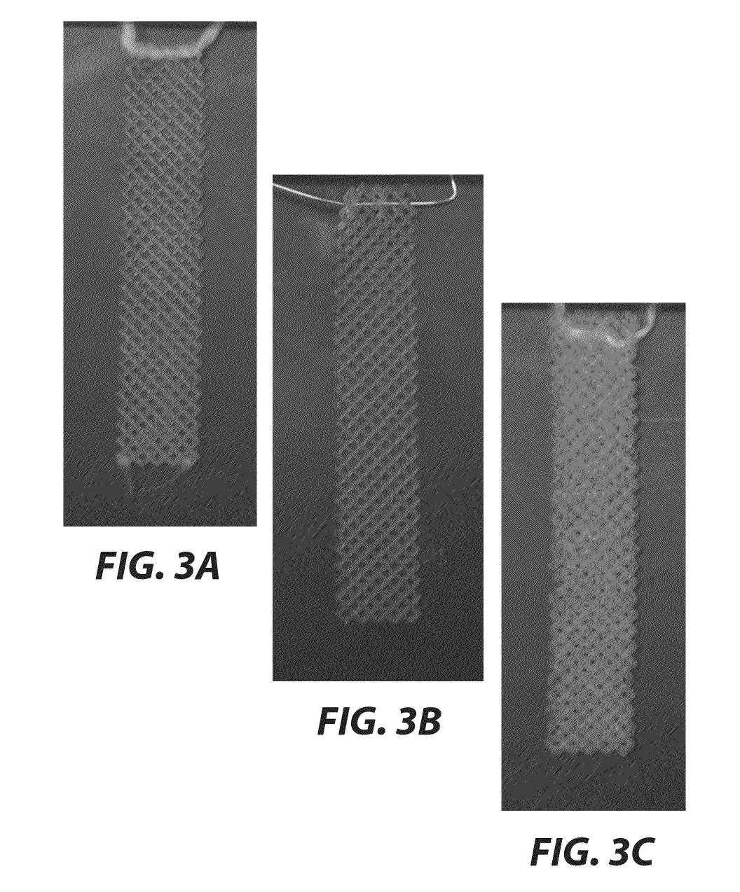

In at least one embodiment, the polymer is thermally polymerizable. Here, the monomer above its glass transition temperature or melting temperature is introduced to a mold that is patterned in relief. The mold is closed forcing the monomer to conform to the relief. The monomer is then exposed to increased temperature such that it polymerizes and retains the shape of the mold upon release. For example, in at least one embodiment, poly(dimethylsiloxane) PDMS may be mixed with gold nanoparticles to produce a “puffy” elastic mesh (FIG. 3C). In yet other embodiments, the polymer comprises swelling polymers and/or hydrogels.

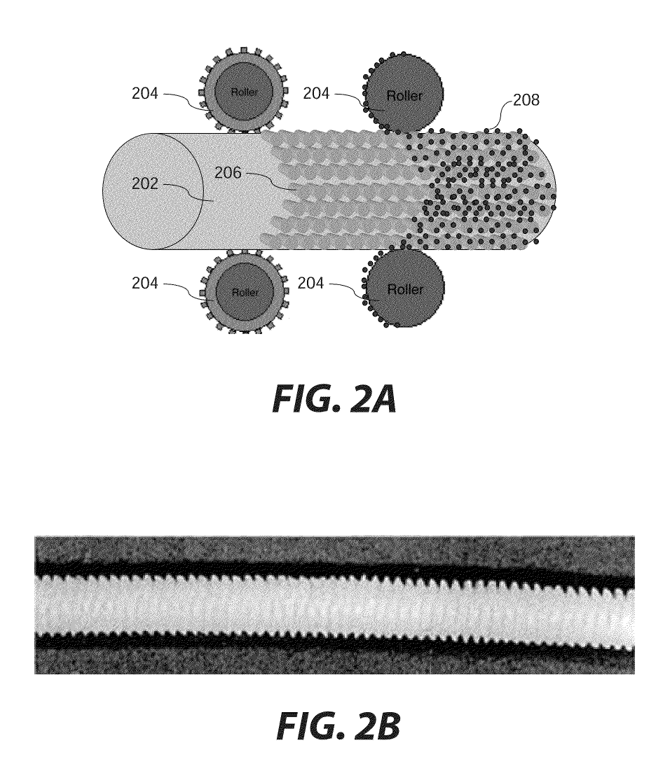

In at least one embodiment, the fibers are formed via extrusion processes. When the Deborah number of the process is of the same order of magnitude or larger than unity, the surface of the extruded fibers will be mechanically unstable such that it develops topology such as sharkskin features (see FIGS. 2A and 2B). In at least one embodiment, the surface of the fiber is extruded with topological patterning including but not limited to axial and spiral configurations.

In some embodiments, the fibers or implements are first made using methods known to those skilled in the art and then the topological relief is added. In at least one embodiment, the topology is made by batch methods comprising, inter alia, imprint lithography, nanoimprint lithography, lithographically induced self assembly, and electrohydrodynamic patterning [14-24]. In several of these embodiments, the polymer is heated above its glass transition temperature, a stamp or mold is placed onto the fiber surface, and the polymer is then quenched. With removal of the stamp, the surface topology remains. In electrohydrodynamic patterning, the polymer (or monomer) is heated above its glass transition temperature and an electric field is applied to a biased (or nonbiased) fiber. The patterning develops as described in the literature before it is quenched by lowering the polymer below its glass transition temperature (or the monomer is polymerized or crosslinked thermally or optically) [14-21].

In at least one embodiment, the fiber or implement topology is added in a high throughput manner using roll-to-roll versions of the same (see FIGS. 2A and 2B).

In some implements, the surgical implements or mesh are formed using 3D printing. For example, a mold may be printed in poly(vinyl alcohol) (PVA), poly(lactic acid) (PLA), or a variety of polymeric materials. The mold may incorporate the desired geometry/topology regular or irregular. In some embodiments, metallic nanoparticles or other particles may be added to the mold and solvent evaporated. In some embodiments, a surface coating of metals or other materials may be added to the mold. The polymer is then added to the mold or polymerized within the mold. In some embodiments, the mold is then dissolved in water leaving the particles or surface coatings exposed on the surface of the implement or mesh. In this manner, the particles or surface coatings can be ensured to reside at the surface.

In at least one embodiment, the topological patterning is comprised of a different material than the remainder of the fiber or surgical implement. In some embodiments, the fibers or surgical implements are first made using methods known to those skilled in the art and then one or more external, outer, or shell layers are added that are patterned in relief. In at least one embodiment, topology is made by batch methods comprising, inter alia, imprint lithography, nanoimprint lithography, lithographically induced self assembly, and electrohydrodynamic patterning. In several of these embodiments, the shell polymer is heated above its glass transition temperature, a stamp or mold is placed onto the fiber surface, and the shell polymer is then quenched. With removal of the stamp, the surface topology on the shell layer remains [17, 22-24]. In electrohydrodynamic patterning, the polymer (or monomer) of the shell is heated above its glass transition temperature and an electric field is applied to a biased (or nonbiased) fiber [14-21]. The patterning of the shell develops as described in the literature before it is quenched by lowering the polymer below its glass transition temperature (or the monomer is polymerized or crosslinked thermally or optically).

In at least one embodiment, the fiber topology is added in a high throughput manner using roll-to-roll versions of the same (see FIGS. 2A and 2B). For example, a fiber 202 may be coated with patterning 206 using rollers 204. Similarly or subsequently, metallic nanoparticles 208 may be added by additional rollers 204.

In some embodiments, the topological patterning is transferred by hot/warm embossing or stamping processes. Here, the material to be transferred is first heated above its glass transition temperature, if necessary. For example, a curved stamp may be impressed, collects the material to be transferred, and then is impressed onto the fiber or surgical implement. In various embodiments, the stamp and surgical implements comprise a variety of distinct but matching geometries. Some portion of the material adheres to the fiber surface. The material is then cooled below its glass transition temperature, photopolymerized, photo crosslinked, thermally polymerized, or thermally crosslinked to secure the material onto the surface of the fiber.

In at least one embodiment, a thin uniform coating of a second shell material is added to the fiber, which itself becomes the core of the new composite fiber. The shell material is heated marginally above its glass transition temperature (e.g., within 5, 10, 15, 20, or 25° C.), if necessary, within a segmented or nonsegmented sheath or enclosure. Portions of the sheath are retracted such that a local portion of the shell material forms bridges between the fiber core and the retracting sheath. If the shell material is a polymer, it is quenched below its glass transition temperature to fix the bridges or spikes in place. Further retraction breaks bridges into spikes if necessary. If the shell material is a monomer or is monomeric, it is then exposed to photon or heat fluxes to drive the shell material to polymerize or crosslink to fix it into place.

In some embodiments, the surgical implement or fiber and any topological patterning, where present, are decorated or coated with humanized or other antibodies specific to collagen, elastin, fibrinin, glycoproteins present in the extracellular matrix, cell surface markers, and so forth. In other embodiments, the surgical implement or fiber and any topological patterning, where present, are decorated or coated with humanized antibody fragments specific to collagen, elastin, fibrinin, glycoproteins present in the extracellular matrix, cell surface markers, and so forth. In yet other embodiments, the surgical implement or fiber and any topological patterning, where present, are decorated or coated with aptamers specific to collagen, elastin, fibrinin, glycoproteins present in the extracellular matrix, cell surface markers, and so forth. In still other embodiments, the surgical implement or fiber and any topological patterning, where present, are decorated or coated with lectins specific to glycoprotein decorated collagen, elastin, fibrinin, glycoproteins present in the extracellular matrix, cell surface markers, and so forth. In further embodiments, the surgical implement or fiber and any topological patterning, where present, are decorated or coated with short protein sequence that preferentially bind to integrins (e.g., RGD sequences) and other protein binding molecular moieties. In other embodiments, the surgical implement or fiber 102 and any topological patterning, where present, are decorated or coated with glycans or sugar molecules 108 specific to glycoconjugate receptors (see FIGS. 1A-1D).

In some embodiments, the surgical implement or fiber and any topological patterning, where present, are decorated or coated with polyethylene glycol (PEG) or other similar surface coatings to minimize protein adsorption and decrease detection by the immune system.

In other embodiments, the surgical implement or fiber and any topological patterning, where present, are decorated or coated with small molecules or polymer termini to provide the required surface charge. DNA molecules are typically negatively charged but cell surfaces may be either positively or negatively charged (though not exclusively) such that fiber surfaces with negative or positive surface charge (i.e., decorated with conjugate bases) are preferred.

In at least one embodiment, the surface of the surgical implement or mesh and any topological patterning, where present, is at least partially coated with ceramic or metallic nanoparticles 106 (see FIGS. 1A-1D). Metals have particularly high Hamaker constants in excess of 20·10⁻²⁰ J [8]. For example, gold and silver are approximately 40·10⁻²⁰ J and 50·10⁻²⁰ J, respectively [13]. In various embodiments, the metallic nanoparticles have diameters less than 10000, 3000, 1000, 900, 800, 700, 600, 500, 400, 300, 200, 100, 90, 80, 70, 60, 50, 40, 30, 20, 10, 9, 8, 7, 6, 5, 4, 3, 2, or 1 nm. In at least one embodiment, the metallic nanoparticles have surface densities comprising multiple monolayers. In at least one embodiment, the metallic nanoparticles have surface densities comprise a single monolayer. In at least one embodiment, the metallic nanoparticles have surface densities less than a monolayer. In at least one embodiment, patterning the surgical implement or fiber surface in topological relief provides additional surface area for nanoparticle display. In at least one embodiment, the nanoparticles also serve as antibacterial agents. For example, silver nanoparticles possess well known antibacterial properties. In at least one embodiment, the nanoparticles are coated with a short oligomeric polymer with a thiol terminus. In some embodiments, the nanoparticles are inert. For example gold nanoparticles are commonly considered to be immunologically inert.

In some embodiments, the nanoparticles are at least partially decorated or at least partially coated with short oligomeric strands of the same material as the patterning, surgical implement or fiber surface. For example, the strands may have a thiol terminus that binds preferentially to metals including but not limited to silver and gold. Similarly, strands may support moieties that can be linked to thiol groups with commonly available chemistries. In at least one embodiment, the nanoparticles are at least partially decorated or at least partially coated with short oligomeric strands or surgical implements of a material that is soluble in the surface of the surgical implement, fiber or the patterning. In at least one embodiment, the patterned strands or surgical implements are first coated with nanoparticles with or without coating before a thermal pulse temporarily raises the surgical implement or fiber surface above its glass transition temperature such that the nanoparticles can at least partially enter the fiber or patterning surface.

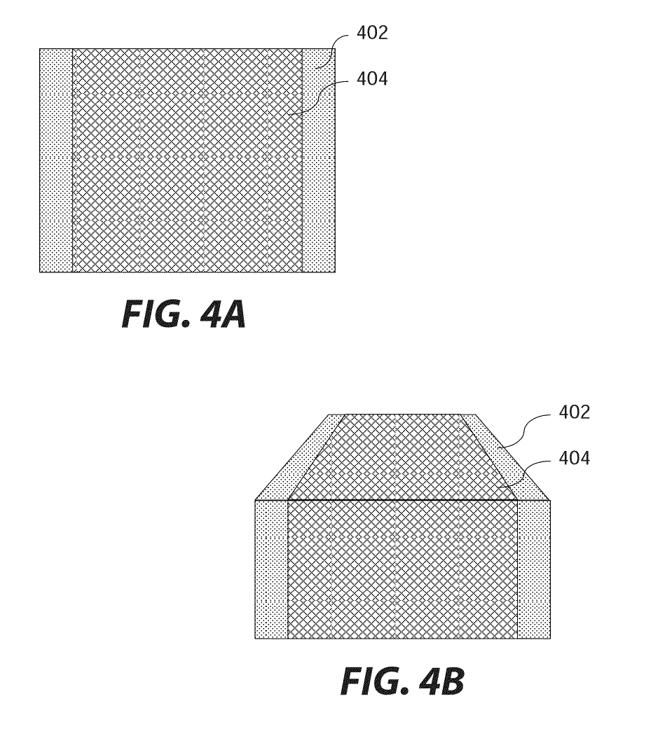

In various embodiments, the disclosed adhesive 402, comprising patterning and/or particles/coatings, is connected to other surgical implements 404 including, but not limited to sutures, mesh, stents, bandages and the like (see FIGS. 4A and 4B) In some embodiments, the adhesive is sutured to surgical mesh before insertion. In some embodiments, the adhesive and mesh are glued (e.g., cyanoacrylates, dermal glues and the like) together. In some embodiments, the adhesive and surgical mesh are taped together or velcroed together so that the anchor with the adhesive remains, but the mesh can be changed repositioned or adjusted. In other embodiments, the adhesive covers a significant portion of the surgical mesh. In some embodiments, the adhesive mesh can be cut using scissors and/or similar implements to cut, shape or contour the mesh so as to adhere to tissue where it is needed.

In various embodiments, the disclosed microadhesive is mechanically stiff. Typically, mesh and related surgical implements should have low stiffness to best mimic the local cellular environment, because mechanically stiff environments may induce the formation of collagen matrices, some of which are avascular leading to erosion, a surgical complication. However, in some embodiments, a small portion of the surgical implement may comprise the microadhesion on a stiffer material to better anchor the mesh or surgical implement to the tissue. For example, a gynecological surgeon may wish to connect mesh to the so called white line in the pelvic region to best anchor the mesh to a solid support. In this case, the edge of the mesh may, for example, comprise both the micropatterning and also the stiffer support to provide immediate (compared to the healing time) adhesion and also induce increased levels of collagen to provide the most robust and stable connection. Stiffer substrates may also be preferential where the tissue can conform to the local patterning and/or embedded surfaces.

In various embodiments, the metallic coating comprises a uniform coating of metals. In some embodiments, the coating is not uniform. In some embodiments, the coating is patchy or incomplete. For example, metals, such as gold, can be applied as thin films, sheets or coatings. In some embodiments, the gold layer is sufficiently thin such that the composite remains flexible [25, 26]. In some embodiments, the gold is applied to less flexible materials using one or more of a variety of methods (e.g., dip coating, deposition, etc.) known to those skilled in the art.

In various embodiments, the softer layers are coupled with stiffer layers or fibers to provide mechanical stability. For instance, a PDMS layer patterned topologically and/or with metallic nanoparticles may be coupled with a thin PLA shim or fibers to provide mechanical strength to the composite. In some embodiments, the PLA is plasticized, blended, or copolymerized.

In some embodiments, the mesh or other surgical implement aim to adhere to one type of tissue without adhering to others. For example, in paravaginal repair of cystocele in which a mesh is used to supplement native fascia, the surgeon connects the mesh to the fascia without connecting it to the adjacent vaginal mucosa. In some embodiments, the surgical implement or mesh comprises the disclosed patterning, particle, and/or metallic coating on one or more of one or more surfaces such that one or more of the surfaces is not patterned, embedded or coated. In some embodiments, the patterning is only on one side. In some embodiments, the patterning is on both sides. In some embodiments, the patterning is on multiple sides. In some embodiments, the patterning is on all sides.

The disclosed nanoparticle decorated fibers overcome a strategic weakness of earlier fiber compositions. The soft flexible nanotopology provides adhesion superior to smooth polymeric fibers. The addition of nanoparticles provides enhanced van der Waals attraction between fiber and tissue relative to gecko fibers, glues, or adhesives because the higher Hamaker constants provide truly maximized van der Waals forces. The use of small protrusions on the fibers is superior to schemes that only utilize the tips of fibers for adhesion. The structures disclosed herein are superior to previous biomimetic gecko-like structures because the disclosed structures provide adhesion in both wet/hydrated and dry environments. Finally, for the purposes of mesh, bandage, and suture design, the disclosed implements and/or fibers are superior to previous art that essentially discloses and enables only gecko-like structures from flat surfaces.

In at least one embodiment, the coating of the surgical implement or mesh comprises a thin monosaccharide or disaccharide layer for ease of insertion. In at least one embodiment, the encoating layer comprises a smooth surface. In at least one embodiment, the layer comprises sugar, sucrose, or other saccharide. For example, the sugar layer begins to dissolve the instant it is hydrated. This provides a thin viscous layer to facilitate smooth and rapid surgical entry of the mesh through narrow and confined incisions. The sugar layer provides a natural separation between the surface charge of the polymer and the tissue to temporarily minimize adhesion. The sugar layer further reduces the van der Waals forces between the tissue and the mesh by decreasing the composite Hamaker constant. A 17% sucrose solution, as the medium, has a Hamaker constant of 10.9·10⁻²⁰ J [11]. For example, the composite Hamaker constant for polymers (3.8·10⁻²⁰ J to 9.5·10⁻²⁰ J) and high Hamaker constant cells (1.4·10⁻¹⁹ J) with water as the media is repulsive and ranges from the 2.7·10⁻²¹ J to 2.5·10⁻²⁰ J. In the presence of a sucrose solution (1.09·10⁻¹⁹ J), this range decreases to −9.8·10⁻²² J to −6.0·10⁻²¹ J. The magnitude shrinks and the sign changes removing the potential for van der Waals attraction and even providing temporary repulsion between the fibers and cellular mass. In each case, the sugar layer temporarily decreases adhesion between the tissue and the mesh. However, the effect is temporary as the sugar layer dissolves and transports away. The duration of this effect depends on the thickness of the sucrose layer, the rate of diffusion, and on the shear rate of the mesh through the body cavity. After the sugar has dissolved away, the adhesion forces due to charge-charge, van der Waals, and biochemical binding act to adhere the mesh to the adjacent tissue as previously disclosed. Similar effects may be achieved by other dissolvable materials at the fiber surface should their Hamaker constants allow. In some embodiments, the dissolvable material is biocompatible.

In an embodiment, the surgical implement or fiber surface comprises a polymer with a Hamaker constant less than or approximately equal to that of water. The Hamaker constant of cells ranges between 8.2·10⁻²¹ to 1.4·10⁻¹⁹ J [11]. To adhere (i.e., have an attractive van der Waals force) to cells with Hamaker constants below that of water (3.2·10⁻²⁰ to 3.7·10⁻²⁰ J), the surface material of the surgical implement or fiber must also have a Hamaker constant below that of water. Otherwise, the van der Waals forces will be repulsive in nature and will minimize adhesion not enhance it. Preferred materials comprise PTFE and ePTFE [9]. In at least one embodiment, the surgical implement or fiber surface is coated with a dilute polyglycan or oligosaccharides. This embodiment leverages the increase of the composite Hamaker constant in the presence of a sucrose solution by organizing the sugar into polymeric or polysaccharide chains. For instance, the composite Hamaker constant for polymers (3.8·10⁻²⁰ J to 9.5·10⁻²⁰ J) and low Hamaker constant cells (8.2·10⁻²¹ J) with water as the media is repulsive and ranges from the −2.6·10⁻²² J to −1.2·10⁻²⁰ J [13]. In the presence of a sucrose solution (1.09·10⁻¹⁹ J), this range increases to 5.3·10⁻²¹ J to 3.2·10⁻²⁰ J. The sign change indicates that with a sugar solution, the van der Waals force is attractive. A similar sign change would be anticipated in the presence of a polysaccharide solution with a similar monomer concentration. In this embodiment, polymers with smaller Hamaker constants are preferred.

The invention disclosed herein further comprises anchors, hooks, and substantially flat, curved or arrayed surfaces that comprise polymeric materials, the surface of which are patterned in relief with topologies similar to those described for surgical implement or fibers above. The invention also comprises decorating the surface of these fixing elements with nanoparticles similar to that described above.

The disclosed adhesive is important for mesh, sutures, stents, and bandages, inter alia. For example, using the adhesive disclosed, surgical mesh can be inserted into smaller spaces. This adhesive removes, minimizes or eliminates the need for extensive dissection to create a larger space for adequate visualization and to accomplish suturing or stapling maneuvers. Less dissection minimizes damage to the underlying tissue. For instance, in the case of paravaginal repair performed trans-vaginally, after separating the vaginal mucosa from the underlying endopelvic fascia, using sharp and blunt dissection to create an approximately one or more millimeter thick space precisely large to enough to accommodate this maneuver. Then the edge of a mesh with the disclosed adhesive material may be pushed to the “white line,” the solid supporting bony structure, using a thin blunt ribbon. This surgical process should not create much tissue trauma or excessive bleeding. Once the mesh edge is against the white line, the sugar covering of the adhesive dissolves, and the adhesive firmly attaches the mesh to the white line. Very minimal operating space is required, with minimal tissue trauma. Using this adhesive strategy to attach the mesh to the white line and the endopelvic fascia is a surface action, with no need for any penetration beyond the surface attachment. This would eliminate all the risks associated with penetrating injuries of the underlying vital structures such as blood vessels, nerves, bowel or urinary tract. This would be applicable, in one example, for the attachment of the mesh to the sacro-spinous ligament, which has nerve and vascular bundles right behind it. If penetration by suturing, staple, hook trocar or anchor of those vital structures occurs during the sacro-spinous ligament, major bleeding, retro-peritoneal hematoma or nerve injury can be substantial complications. Using the disclosed adhesive attachment to attach mesh will avoid these possible complications. This approach can be applied to the examples of minimally invasive mastopexy and endoscopic abdominoplasty among others, for instance.

Indeed, the disclosed invention solves the general problem of strongly but removably attaching surfaces in liquid rich environments or aqueous environments in specific. Indeed, it allows for gecko adhesives or gecko glues that operate in hydrated environments where they currently fail.

### EXAMPLE

An aluminum mold was prepared by machining grooves approximately 2 mm deep in a diamond pattern. Gold or silver nanoparticles in suspension nominally 50 nm in diameter, for example, from Ted Pella were placed in the mold (approximately 1 mL of 4.5·10⁻¹⁰ particles/mL for the gold) and the liquid allowed to evaporate until dry so that a mostly uniform thin film was prepared. A control sample was prepared without addition of gold nanoparticles. To both the sample and control, a poly(dimethylsiloxane) (PDMS) from Dow Corning called Silastic 4210 was prepared according to manufacturer's instructions and, while still a liquid, carefully placed in the mold with a syringe. The aluminum mold and its contents were then incubated according to manufacturer's instructions such that solid elastic PDMS mesh with or without metallic nanoparticles on one surface were prepared. The mesh were then carefully removed from the aluminum mold with a combination of tension and a prying implement. The mesh containing gold nanoparticles clearly displayed a red tint, while the control sample had a whitish opaque coloring.

Chicken skin was obtained commercially. One layer of chicken skin clearly larger than any of the three mesh individually was first laid down. A selected mesh was placed on the first chicken skin layer. A second chicken skin layer was then placed over the mesh such that the mesh was sandwiched between the two layers of chicken skin. The mesh was threaded or connected with a thin wire or thread to a weight such that the mesh was horizontal, but the weight remained vertical. The magnitude of the weight was increased until the mesh slipped between two layers of chicken skin. The largest weight and time from initiation of slippage until the mesh was completely released was recorded. The weight of the portion of the chicken skin directly over the mesh was approximately 2.6 g and the weight of the mesh was approximately 0.87 g.

Qualitatively, the 8.0 cm long mesh without gold nanoparticles clearly sustained much less weight than the mesh with metallic nanoparticles. For example, the control mesh (FIG. 3A) only began to fail at 54 g of weight and released completely from the chicken skin in 6.9 s. In contrast, the mesh with gold nanoparticles embedded in its surface (FIG. 3B) sustained 80 g of weight and then released completely over 30.9 s. Clearly including the metal particles substantially increases the weight sustained and the release time as disclosed herein.

A coefficient of friction, μ, can be estimated by balancing the change in potential energy against the developed kinetic energy and friction losses as

$$μ = \frac{{m}_{w}}{{m}_{m} + 2 {m}_{t}} ( 1 - \frac{{v}^{2}}{2 g L} ) , \qquad ( 1 )$$

where m_{w} is the mass of the weight, mₘ is the mass of the mesh, mᵢ is the mass of the tissue, v is the developed velocity, g is the gravitational constant and L is the length of the mesh for those with adhesive on both sides. The static coefficient derived from a force balance returns Eq. 1 with v=0. The coefficient of friction was approximate 9 for the control, whereas with metallic nanoparticles embedded in the surface the coefficient of friction increases to 13. This represents a 50% increase. Similar increases on stiffer polymers (e.g., PLA or copolymers thereof) would be expected. Although many coefficients of friction often remain between zero and one, coefficients of friction for rubbers often significantly exceed unity. Indeed, coefficients of friction larger than unity simply imply that the friction forces exceeded the normal force; there are no inherent thermodynamic limits to preclude coefficient of frictions exceeding unity or even ten.

Experimentally, the degree of adhesion is much greater in liquid environments than in dry environments. Increasing the surface area also has an effect. For example, mixing the metallic nanoparticles into the bulk of the PDMS before incubation decreases the amount of metallic nanoparticle near the surface but dramatically increase the volume of the PDMS mesh with corresponding increases in the surface area (see FIG. 3C). Higher incubation rates increase the volume increase. This gives more surface area for contact, which though it should not make much difference according to Amontons' law, can partially but not completely compensate for the loss in particle density.

From the foregoing, it will be appreciated that specific embodiments of the disclosure have been described herein for purposes of illustration, but that various modifications may be made without deviating from the spirit and scope of the disclosure. Aspects described in the context of particular embodiments may be combined or eliminated with other embodiments. Further, although advantages associated with certain embodiments have been described in the context of those embodiments, other embodiments may also exhibit such advantages, and not all embodiments need necessarily exhibit such advantages to fall within the scope of the disclosure. Accordingly, the disclosure is not limited by the appended claims.

### REFERENCES CITED

[1] Osswald T A. Polymer Processing Fundamentals Cincinnati, Ohio: Hanser Gardner Publications; 1998.
[2] Kendall K, Rehfeldt F, Kendall M. Adhesion of Cells, Viruses and Nanoparticles. Dordrecht: Springer; 2010.
[3] Savia M, Zhou Q. Van der Waals force computation of freely oriented rough surfaces for micromanipulation purposes. 2010 IEEE/RSJ International Conference on Intelligent Robots and Systems (IROS). Taipei: IEEE; 2010.
[4] http://robotics.eecs.berkeley.edu/˜ronf/Gecko/index.html. Accessed Sep. 13, 2012.
[5] http://robotics.eecs.berkeley.edu/˜ronf/Gecko/gecko-compare.html. Accessed Sep. 13, 2013.
[6] Gregory J. Particles in water: properties and processes. Boca Raton, Fla.: CRC Press; 2005.
[7] Elimelech M, Gregory J, Jia X, Williams R. Particle deposition and aggregation: measurement, modelling and simulation, by Woburn, Mass.: Butterworth-Heinemann; 1998.
[8] Cosgrove T. Colloid Science: Principles, Methods and Applications. New York: Wiley-Blackwell; 2010.
[9] http://courses.washington.edu/overney/Material/HandoutsChemE498/Hamaker_Constant.pdf. Accessed on Sep. 13, 2013.
[10] Crowe C T. Multiphase Flow Handbook. Boca Raton, Fla.: CRC Press; 2005.
[11] Xia Z, Goldsmith H L, Ven TGMvd. Kinetics of Specific and Nonspecific Adhesion of Red Blood Cells on Glass. Biophysical Journal. 1993;65:1073-83.
[12] Dengis P B, Nelissen L R, Rouxhet P G. Mechanisms of Yeast Flocculation: Comparison of Top- and Bottom-Fermenting Strains. Applied and Environmental Microbiology. 1995;61:718-28.
[13] Ahmadi G. http://web2.clarkson.edu/projects/crcd/me437/downloads/5_vanderWaals.pdf Accessed on Sep. 13, 2013.
[14] Pease L F, Russel W B. Charge Driven, Electrohydrodynamic Patterning of Thin Films. Journal of Chemical Physics. 2006;125:184716.
[15] Wu N, Pease L F, Russel W B. Toward Large-scale Alignment of Electrohydrodynamic Patterning of Thin Polymer Films. Advanced Functional Materials. 2006;16:1992-9.
[16] Wu N, Pease L F, Russel W B. Electric-field Induced Thin Polymer Film Patterns: weakly nonlinear and fully nonlinear evolution. Langmuir. 2005;21:12290-302.
[17] Deshpande P, Pease L F, Chen L, Chou S Y, Russel W B. Cylindrically Symmetric Electrohydrodynamic Patterning. Physical Review E. 2004;70:041601.
[18] Pease L F, Russel W B. Limitations on Length Scales for Electrostatically Induced Submicron Pillars and Holes. Langmuir. 2004;20:795-804.
[19] Pease L F, Deshpande P, Chou S Y, Russel W B. Polymeric Gratings via Fracture with Deep Sub-micron Length Scales and Large Areas. Proceedings of the XIVth International Congress on Rheology. 2004:Art. No. MP13.
[20] Pease L F, Russel W B. Electrostatically Induced Submicron Patterning of Thin Perfect and Leaky Dielectric Films: A generalized linear stability analysis. Journal of Chemical Physics. 2003;118:3790-803.
[21] Pease L F, Russel W B. Linear Stability Analysis of Thin Leaky Dielectric Films Subjected to Electric Fields. Journal of Non-Newtonian Fluid Mechanics. 2002;102:233-50.
[22] Chou S Y, Krauss P R, Renstrom P J. Nanoimprint Lithography. J Vac Sci and Technol, B. 1996;14:1-5.
[23] Chou S Y, Krauss P R, Zhang W, Guo L, Zhuang L. Sub-10 nm Imprint Lithography and Applications. J Vac Sci Technol, B. 1997;15:2897-904.
[24] Tan H, Gilbertson A, Chou S Y. Roller nanoimprint lithography. J Vac Sci and Technol, B. 1998;16:3926-8.
[25] Graz I M, Cotton D P J, Robinson A, Lacour S P. Silicone substrate with in situ relief for stretchable thin-film transistors. Applied Physics Letters. 2011;98:124101.

[26] Lacour S P, Wagner S, Huang Z, Suo Z. Stretchable gold conductors on elastomeric substrates Applied Physics Letters. 2003;82:2404-6.

The subject matter disclosed in each of the foregoing references is incorporated by reference herein.

The embodiments of the invention in which an exclusive property or privilege is claimed are defined as follows:

1. An adhesive surface, comprising at least a nanoparticle coating.

2. The adhesive surface of claim 1, wherein the surface is adhesive to tissue in a living body.

3. The adhesive surface of claim 1, wherein the nanoparticle coating is metallic.

4. The adhesive surface of claim 3, wherein the nanoparticle coating comprises gold or silver.

5. The adhesive surface of claim 1, wherein the adhesive surface constitutes one or more elements of a bandage or surgical mesh.

6. The adhesive surface of claim 1, wherein the adhesive surface constitutes a surface of a suture.

7. The adhesive surface of claim 1, wherein the adhesive surface is sized, shaped, and configured for surgical application in a body.

8. The adhesive surface of claim 1, wherein the surface further comprises a dissolvable coating.

9. The adhesive surface of claim 8, wherein the dissolvable coating comprises a polysaccharide or oligosaccharide.

10. The adhesive surface of claim 8, wherein the dissolvable coating comprises sucrose.

11. An adhesive surface, comprising at least a metallic coating.

12. The adhesive surface of claim 11, wherein the metallic coating comprises gold or silver.

13. The adhesive surface of claim 11, wherein the adhesive surface constitutes one or more elements of a bandage or surgical mesh.

14. The adhesive surface of claim 11, wherein the adhesive surface constitutes a surface of a suture.

15. The adhesive surface of claim 11, wherein the adhesive surface is sized, shaped, and configured for surgical application in a body.

16. An adhesive surface, comprising at least a fiber with a micro/nano patterned topology.

17. The adhesive surface of claim 16, wherein the fiber has a surface that is patterned in topological relief.

18. The adhesive surface of claim 17, wherein the fiber has an axis, and wherein the patterning comprises at least one of ridges substantially parallel to the fiber axis, ridges substantially perpendicular to the fiber axis, ridges that spiral around the fiber, cylindrical protrusions substantially normal the fiber surface, undulations of the fiber surface, or random asperities of the fiber surface.

19. The adhesive surface of claim 18, wherein the patterning is less than 700 nm.

20. The adhesive surface of claim 18, wherein the patterning is greater than 10 microns.

21. The adhesive surface of claim 16, wherein the fiber is encased in a dissolvable coating.

22. The adhesive surface of claim 21, wherein the dissolvable coating comprises a polysaccharide or oligosaccharide.

23. The adhesive surface of claim 21, wherein the dissolvable coating comprises sucrose.

24. The adhesive surface of claim 16, wherein the adhesive surface constitutes one or more elements of a bandage or surgical mesh.

25. The adhesive surface of claim 16, wherein the adhesive surface constitutes a surface of a suture.

26. The adhesive surface of claim 16, wherein the adhesive surface is sized, shaped, and configured for surgical application in a body.

27. The adhesive surface of claim 21, further comprising a nanoparticle coating.
